(19) United States
(12) Patent Application Publication
(10) Pub. No.: US 2009/0286863 A1
(43) Pub. Date: Nov. 19, 2009

(54) SULFAMATOBENZOTHIOPHENE DERIVATIVES

(76) Inventors: David Bruge, Frankfurt (DE); Wolfgang Staehle, Ingelheim (DE); Gerald Scholz, Bensheim (DE); Benoit Rondot, La Colle Sur Loup (FR); Jean Lafay, Nice (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/307,311

(22) PCT Filed: Jun. 5, 2007
(86) PCT No.: PCT/EP07/04962

§ 371 Date: Jan. 2, 2009

(30) Foreign Application Priority Data
Jul. 5, 2006 (EP) 06013922.7

(51) Int. Cl. A61K 31/381; C07D 333/54; A61P 37/00; A61P 25/28

(52) U.S. Cl. 514/443; 549/51; 549/53

(57) ABSTRACT

The invention relates to novel compound of the general formula (I), in which R has the meanings indicated in Claim 1, to the preparation thereof and to the use thereof as medicaments. The compounds (I) are inhibitors of steroid sulfatase and are used for the treatment of cancer.

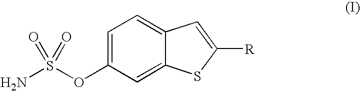

The invention relates to novel compounds of the formula (I)

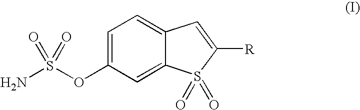

in which
R is cycloalkyl-(CH₂)ₘ—, (AA′R¹)C—(CH₂)ₙ—, cycloalkylidene-(CH₂)ₙ—, cycloalkyl-(CH₂)ₒCO— or cycloalkyl-(CH₂)ₒC(OH)—
R¹ denotes A or H,
A, A′ is each, independently of one another, alkyl having 1 to 4 C atoms
m denotes 2, 3 or 4,
n denotes 1, 2, 3 or 4,
o denotes 0, 1, 2 or 3,
   and pharmaceutically usable derivatives, salts, solvates and tautomers thereof, including mixtures thereof in all ratios.

The invention was based on the object of finding novel compounds having valuable properties, in particular those which are used for the preparation of medicaments.

It has been found that the compounds of the formula (I) and salts and/or solvates thereof have very valuable pharmacological properties while being well tolerated.

Compounds having a similar structure are disclosed in WO2004/101545 A1, where all compounds are inhibitors of steroid sulfatase.

The enzyme steroid sulfatase (E.C. 3.1.6.2., STS) catalyses the hydrolysis of oestrone sulfate to oestrone and of DHEA sulfate to DHEA (Dibbelt L, Biol. Chem, Hoppe-Seyler, 1991, 372, 173-185 and Stein C, J. Biol. Chem., 1989, 264, 13865 13872).

The steroid sulfatase pathway has been the focus of recent attention in the context of breast cancer, with regard to the local intra-tissue formation of oestrogens from the abundant circulating pool of oestrone sulfate (E₁S) (Pasqualini J R, J. Steroid Biochem. Mol. Biol., 1999, 69, 287-292 and Purohit A, Mol. Cell. Endocrinol., 2001, 171, 129-135).

Inhibition of this enzyme would inhibit the formation of free oestrone (E₁) from E₁S, (E₁) can be transformed into oestradiol (E₂) by enzymatic reduction. In addition to the oestrone sulfatase pathway, it is now believed that another potent oestrogen, androstenediol (adiol) obtained from DHEA after hydrolysis of DHEA sulfate, could be another important route, in the support of growth and development of hormone-dependent breast tumours.

In patients with hormone-dependent cancers, aromatase inhibitors are currently used to prevent oestrogen synthesis. However, clinical trials showed a relative lack of efficacy for patients with oestrogen receptor-positive tumours (Castiglione-Gertsch M, Eur. J. Cancer, 1996, 32A, 393-395 and Jonat W, Eur. J. Cancer, 1996, 32A, 404-412). As an explanation, the steroid sulfatase pathway could be another important route for oestrogen formation in breast tumours.

EMATE (Ahmed S. Curr. Med. Chem., 2002, 9, 2, 263-273), oestrone-3-sulfamate, is the classical standard steroid sulfatase inhibitor but with the major drawback of being oestrogenic because of its mechanism of inhibition: the sulfamate moiety is cleaved off during the enzyme deactivation process, which releases E₁ not from E₁S, but from EMATE itself (Ahmed S. J. Steroid Biochem. Mol. Biol., 2002, 80, 429-440).

Other non-steroidal sulfamate compounds which release derivatives without oestrogenic properties are presented as acceptable drug candidates, in particular 6,6,7-COUMATE, a standard non-oestrogenic sulfatase inhibitor from the literature (Purohit A, Cancer Res., 2000, 60, 3394-3396).

Accordingly, there is a need for steroid sulfatase inhibitors with regard to the treatment of, in particular, oestrogen-dependent diseases.

The invention also relates to the hydrates and solvates of these compounds. Solvates of the compounds are taken to mean adductions of inert solvent molecules onto the compounds which form owing to their mutual attractive force. Solvates are, for example, mono- or dihydrates or alcoholates.

Pharmaceutically usable derivatives are taken to mean, for example, the salts of the compounds according to the invention and also so-called prodrug compounds. Prodrug derivatives are taken to mean compounds of the formula (I) which have been modified by means of, for example, alkyl or acyl groups, sugars or oligopeptides and which are rapidly cleaved in the organism to form the effective compounds according to the invention. These also include biodegradable polymer derivatives of the compounds according to the invention, as described, for example, in Int. J. Pharm. 115, 61-67 (1995).

The expression “effective amount” denotes the amount of a medicament or of a pharmaceutical active compound which causes in a tissue, system, animal or human a biological or medical response which is sought or desired, for example, by a researcher or physician. In addition, the expression “therapeutically effective amount” denotes an amount which, compared with a corresponding subject who has not received this amount, has the following consequence:

improved treatment, healing, prevention or elimination of a disease, syndrome, condition, complaint, disorder or side-effects or also the reduction in the advance of a disease, complaint or disorder. The expression “therapeutically effective amount” also encompasses the amounts which are effective for increasing normal physiological function.

The invention relates to the compounds of the formula (I) and salts thereof and to a process for the preparation of compounds of the formula (I) and pharmaceutically usable derivatives, salts and solvates thereof, characterised in that
a) a compound of the general formula (II)

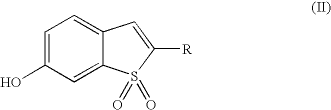

in which R has the meaning given in the general formula (I) according to claim 1,
is reacted with sulfamoyl chloride (H₂N—SO₂—Cl);
or
b) a compound of the general formula (III)

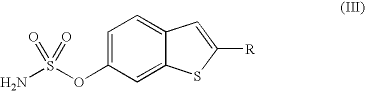

in which R has the meaning given in the general formula (I) according to claim 1,
is oxidised;
and/or
c) a compound of the general formula (I) is converted into one of its salts.

Above and below, the radicals R, R¹ A and A′ have the meanings indicated for the formula (I), unless expressly indicated otherwise.

A, A′ denote, in each case independently of one another, alkyl having 1, 2, 3 or 4 C atoms, is unbranched (linear) or branched, and is preferably methyl, ethyl, n-propyl, isopropyl, butyl, isobutyl, sec-butyl or tert-butyl, furthermore trifluoromethyl, pentafluoroethyl or 1,1,1-trifluoroethyl. Methyl is particularly preferred.

Cycloalkyl has 3 to 8 C atoms and denotes cyclopropyl, cyclobutyl, cyclopentyl, cyclohexyl, cycloheptyl or cyclooctyl, preferably cyclopentyl, cyclohexyl or cycloheptyl, particularly preferably cycloheptyl or cyclohexyl.

Cycloalkylidene has 3 to 8 C atoms and denotes cyclopropylidene, cyclobutylidene, cyclopentylidene, cyclohexylidene, cycloheptylidene or cyclooctylidene, preferably cyclopentylidene, cyclohexylidene or cycloheptylidene, particularly preferably cyclohexylidene.

Throughout the invention, all radicals which occur more than once may be identical or different, i.e. are independent of one another.

m is 2, 3 or 4, preferably 2 or 3 and very particularly preferably 2.
n is 1, 2, 3 or 4, preferably 1 or 2 and very particularly preferably 1.
o is 0, 1, 2, or 3, preferably 0, 1 or 2 and very particularly preferably 0.

Accordingly, the invention relates, in particular, to the compounds of the formula (I) in which at least one of the radicals mentioned has one of the preferred meanings indicated above. Some preferred groups of compounds can be expressed by the following sub-formulae Ia to Ik, which conform to the formula (I) and in which the radicals not designated in greater detail have the meaning indicated for the formula (I), but in which
in Ia R is cyclohexyl-(CH₂)ₘ— or cycloheptyl-(CH₂)ₘ— and
   m is 2 or 3;
in Ib R is (AA′R¹)C—(CH₂)ₙ—,
   R¹ is A or H,
   A, A′ is each, independently of one another, alkyl having 1 and/or 2 C atoms,
   n is 1 or 2;
in Ic R is cyclohexylidene-(CH₂)ₙ— or cycloheptylidene-(CH₂)ₙ— and
   n is 1 or 2;
in Id R is cyclohexyl-(CH₂)ₒCO— or cycloheptyl-(CH₂)ₒCO— and
   o is 0 or 1;
in Ie R is cyclohexyl-(CH₂)ₒC(OH)— and
   o is 0 or 1;
      and pharmaceutically usable derivatives, salts, solvates and tautomers thereof, including mixtures thereof in all ratios.

The compounds of the formula (I) and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants known per se which are not mentioned in greater detail here.

Compounds of the formula (I) can preferably be obtained by reacting compounds of the formula (II) with sulfamoyl chloride or oxidising compounds of the formula (III).

The compounds of the formula (II) and of the formula (III) are generally known. If they are novel, however, they can be prepared by methods known per se.

The reaction of the compounds of the formula (II) with sulfamoyl chloride carried out in an inert solvent.

Depending on the conditions used, the reaction time is between a few minutes and 14 days, the reaction temperature is between about −15° and 150°, normally between 5° and 30°, particularly preferably between 10° and 15° C.

Suitable inert solvents are, for example, hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide (DMA) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents. Dimethylacetamide (DMA) is particularly preferred.

Oxidations, in particular the oxidation of compounds of the formula (III) to give compounds of the formula (I), are carried out by methods known to the person skilled in the art. A standard method is the oxidations using hydrogen peroxide in trifluoroacetic acid (TFA), for example under conditions as described by Grivas and Ronne (Acta Chemica Scandinavia 49, 225-229 (1995)).

The cleavage of an ether is carried out using methods as are known to the person skilled in the art. A standard method for ether cleavage, for example of a methyl ether, is the use of boron tribromide (BBr₃), for example under conditions as described by McOmie (Tetrahedron, 24, 2289-2292 (1968)).

Pharmaceutical Salts and Other Forms
The said compounds according to the invention can be used in their final non-salt form. The present encompasses invention also the use of these compounds in the form of their pharmaceutically acceptable salts, which can be derived from various organic and inorganic acids and bases by procedures known in the art. Pharmaceutically acceptable salt forms of the compounds of the formula (I) are for the most part prepared by conventional methods.

In the case of certain compounds of the formula (I), acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds of the formula (I) include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.
Furthermore, the base salts of the compounds according to the invention include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline-earth metal salts calcium and magnesium. Salts of the compounds of the formula (I) which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris(hydroxymethyl)methylamine (tromethamine), but this is not intended to represent a restriction.
Compounds of the present invention which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.
The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.
The acid-addition salts of basic compounds of the formula (I) are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.
As mentioned, the pharmaceutically acceptable base-addition salts of the compounds of the formula (I) are formed with metals or amines, such as alkali metals and alkaline-earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.
The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.
If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.
With regard to that stated above, it can be seen that the expression “pharmaceutically acceptable salt” in the present connection is taken to mean an active compound which comprises a compound of the formula (I) in the form of one of its salts, in particular if this salt form imparts improved pharmacokinetic properties on the active compound compared with the free form of the active compound or any other salt form of the active compound used earlier. The pharmaceutically acceptable salt form of the active compound can also provide this active compound for the first time with a desired pharmacokinetic property which it did not have earlier and can even have a positive influence on the pharmacodynamics of this active compound with respect to its therapeutic efficacy in the body.

The invention furthermore relates to medicaments comprising at least one compound according to the invention and/or pharmaceutically usable derivatives, salts, solvates and tautomers thereof including mixtures thereof in all ratios, and optionally excipients and/or adjuvants.

Pharmaceutical formulations can be administered in the form of dosage units which comprise a predetermined amount of active compound per dosage unit. Such a unit can comprise, for example, 0.1 mg to 3 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active compound per dosage unit. Preferred dosage unit formulations are those which comprise a daily dose or part-dose, as indicated above, or a corresponding fraction thereof of an active compound. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active compound with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-compound component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape which are broken up to form granules. The granules can be lubricated by addition of stearic acids a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds according to the invention and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention and the salts, solvates and physiologically functional derivatives thereof can also be delivered using monoclonal antibodies as individual carriers to which the compound molecules are coupled. The compounds can also be coupled to soluble polymers as targeted medicament supports. Such polymers may include polyvinylpyrrolidone, pyran copolymer, polyhydroxypropylmethacrylamidophenol, polyhydroxyethylaspartamidophenol or polyethylene oxide polylysine, substituted by palmitoyl radicals, The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, poly-dihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active compound can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active compound can be employed either with a paraffinic or a water-miscible cream base. Alternatively, the active compound can be formulated to give a cream with an oil-in-water cream base or a water-in-oil base.

Pharmaceutical formulations adapted for topical application to the eye include eye drops, in which the active compound is dissolved or suspended in a suitable vehicle, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the vehicle is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as vehicle include active-compound solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound according to the invention depends on a number of factors, including, for example, the age and weight of the human or animal, the precise condition requiring treatment, and its severity, the nature of the formulation and the method of administration, and is ultimately determined by the treating doctor or vet. However, an effective amount of a compound according to the invention for the treatment is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of the other conditions mentioned above.

The invention furthermore relates to medicaments comprising at least one compound according to the invention and/or pharmaceutically usable derivatives, salts, solvates and tautomers thereof, including mixtures thereof in all ratios, and at least one further medicament active compound.

The invention also relates to a set (kit) comprising separate packs of
(a) an effective amount of a compound of the formula (I) and/or pharmaceutically usable derivatives, solvates and tautomers thereof, including mixtures thereof in all ratios, and
(b) an effective amount of a further medicament active compound.

The set comprises suitable containers, such as boxes, individual bottles, bags or ampoules. The set may, for example, comprise separate ampoules, each containing an effective amount of a compound according to the invention and/or pharmaceutically usable derivatives, solvates and tautomers thereof, including mixtures thereof in all ratios, and an effective amount of a further medicament active compound in dissolved or lyophilised form.

### Use

The present compounds are suitable as pharmaceutical active compounds for mammals, in particular for humans, in the treatment of diseases in which steroid sulfatase plays a role.

The invention thus relates to the use of compounds according to the invention, and pharmaceutically usable derivatives, solvates and tautomers thereof, including mixtures thereof in all ratios, for the preparation of a medicament for the treatment of diseases in which the inhibition, regulation and/or modulation of steroid sulfatase plays a role.

In view of their ability to inhibit steroid sulfatase and thus to dry out other sources of endogenous oestrogens in contrast to aromatase inhibitors, the compounds according to the invention can be used alone or in combination with one or more other sexual hormone therapeutic agents, such as anti-oestrogens, SERMs (selective oestrogen receptor modulators), anti-aromatases, anti-androgens, lyase inhibitors, progestins or LH-RH agonists or antagonists, for the treatment or prevention of oestrogen-dependent disorders or diseases. The compounds according to the invention can also be used for the control or management of oestrogen-regulated reproductive functions, such as male or female fertility, pregnancy, abortion or delivery in humans as well as wild or domestic animal species, alone or in combination with one or more other therapeutic agents, such as LH-RH agonists or antagonists, oestroprogestative contraceptives, progestins, antiprogestins or prostaglandins.

Since the breasts are sensitive targets of oestrogen-stimulated proliferation and/or differentiation, the compounds according to the invention can be used for the treatment or prevention of benign breast diseases in women, gynaecomastia in men and benign or malignant breast tumours with or without metastasis both in men and women or in male or female domestic animals. The compounds according to the invention can furthermore be used for the treatment or prevention of benign or malignant diseases of the uterus or ovaries. In each case, the compounds according to the invention can be used alone or in combination with one or more other sexual hormone therapeutic agents, such as those mentioned above. The invention therefore also relates to the use of the compounds of the formula (I) and pharmaceutically usable derivatives, salts, solvates and tautomers thereof, including mixtures thereof in all ratios, for the preparation of a medicament for the treatment or prevention of benign or malignant diseases of the breast, uterus or ovaries, optionally also in combination with one or more active compounds selected from the group of the anti-oestrogens, SERMs, aromatase inhibitors, anti-androgens, lyase inhibitors, gestagens and LH-RH agonists and antagonists.

Since the enzyme steroid sulfatase transforms DHEA sulfate into DHEA, a precursor of active androgens (testosterone and dihydrotestosterone), the compounds according to the invention can be used for the treatment or prevention of androgen-dependent diseases, such as androgenic alopecia (male pattern loss), (Hoffman R et al., J. Invest. Dermatol., 2001, 117, 1342-1348) or acne (Billich A et al., 1999, WO 9952890), benign or malignant diseases of the prostate or testes (Reed M J, Rev. Endocr. Relat. Cancer, 1993, 45, 51-62), alone or in combination with one or more other sexual hormone therapeutic agents, such as antiandrogens, anti-oestrogens, SERMs, antiaromatase, progestins, lyase inhibitors or LH-RH agonists or antagonists. invention therefore furthermore relates to the use of compounds of the formula (I) and pharmaceutically usable derivatives, salts, solvates and tautomers thereof including mixtures thereof in all ratios, for the preparation of a medicament for the treatment or prevention of benign or malignant diseases of the prostate or testes, optionally also in combination with one or more active compounds selected from the group of the anti-oestrogens, SERMs, aromatase inhibitors, antiandrogens, lyase inhibitors, gestagens and LH-RH agonists and antagonists.

Inhibitors of steroid sulfatase are also potentially involved in the treatment of cognitive dysfunction as they are able to enhance learning and spatial memory in rats (Johnson D A, Brain Res, 2000, 865, 286-290). DHEA sulfate as a neurosteroid affects a number of neurotransmitter systems, including those involving acetylcholine, glutamate and GABA, resulting in increased neuronal excitability (Wolf O T, Brain Res. Rev, 1999, 30, 264-288). The invention therefore also relates to the use of the compounds of the formula (I) and pharmaceutically usable derivatives, salts, solvates and tautomers thereof, including mixtures thereof in all ratios, for the preparation of a medicament for the treatment or prevention of cognitive dysfunction.

In addition, oestrogens are involved in the regulation of the balance between Th₁ and Th₂ predominant immune functions and may therefore be suitable for the treatment or prevention of gender-dependent autoimmune diseases, such as lupus erythematosus, multiple sclerosis, rheumatoid arthritis and the like (Daynes R A, J. Exp. Med. 1990, 171, 979-996). Steroid sulfatase inhibition has been shown to be protective in models of contact allergy and collagen-induced arthritis in rodents (Suitters A J, Immunology, 1997, 91, 314-321). The invention therefore also relates to the use of the compounds of the formula (I) and pharmaceutically usable derivatives, salts, solvates and tautomers thereof, including mixtures thereof in all ratios, for the preparation of a medicament for the treatment or prevention of immune diseases.

Studies using 2-MeOEMATE have shown that steroid sulfatase inhibitors have a potent oestradiol-independent growth-inhibitory effect (MacCARTHY MOOROGH L, Cancer Research, 2000, 60, 5441-5450). Surprisingly, a decrease in tumour volume was observed with the compounds according to the invention, with low tumour steroid sulfatase inhibition. In view of this, the compounds according to the invention could lead to a decrease in cellular division because of the large interaction between such new chemical entities and the microtubular network within the cancerous cell, whatever the tissue, including breast, endometrium, uteri, prostate, testis or metastasis generated from. The compounds according to the invention could therefore be suitable for the treatment of non-oestrogen-dependent cancer.

Accordingly, it is a further object of the invention to provide a method for the treatment of the above-mentioned diseases or disorders, in particular oestrogen-dependent diseases or disorders, i.e. oestrogen-induced or oestrogen-stimulated diseases or disorders (GOLOB T. Bioorg. Med. Chem., 2002, 10, 3941-3953). The method comprises administering a therapeutically effective amount of a compound of the formula (I) to a subject (human or animal) in need thereof.

### Test Methods for Measurement of Steroid Sulfatase Inhibitors

### Measurement of the Inhibition of Sulfatase Activity in JEG3 Cells (According to Duncan 1993)

Principle: The human chorion carcinoma cell line JEG3 constitutively expresses high amounts of steroid sulfatase and can therefore be used for the determination of the inhibition of cellular sulfatase activity. To this end, the substrate of sulfatase, oestrogen sulfate, is added to the cells in a defined physiological concentration, and the amount of the product formed, the oestrone and oestradiol concentration are measured.
Method: JEG3 cells are sown in 96-well plates in a density of about 1×10⁵ cells/well in MEM plus 10% of FCS. At about 80% confluence, the cells are washed with PBS, and the test substances are added in a concentration series and 5 nM radioactive ³H-E₁S in DMEM. After an incubation time of 4 hours at 37° C., 100 μl of the incubation medium is removed and transferred into another 96-well plate. For extraction of the radioactive products E1 and E2 formed, 300 μl of toluene is added. After shaking for 30 seconds and centrifugation, the toluene phase is removed and evaporated overnight with liquid nitrogen. Next day, 100 μl of ethanol is added, the mixture is shaken, and 150 μl of scintillation liquid is added, and the radioactivity is determined.
Reference: DUNCAN L., PUROHIT A., HOWARTH M., POTTER R. V. L. and REED M. J. Inhibition of estrone sulfatase activity by estrone-3-methylthiophosphonate: a potential therapeutic agent in breast cancer. Cancer Research, 1993, 53: 298-303.

### Measurement of the Inhibition of Alkaline Phosphatase in Ishikawa Cells (Littlefield 1990)

Principle: In the Ishikawa human endometrium tumour cell line, the induction of alkaline phosphatase is used as a marker for the oestrogenic activity of test substances. The basis for this is regulation of the alkaline phosphatase gene via the oestrogen receptor and thus via oestrogens. The addition of substances having oestrogenic activity causes induction of the alkaline phosphatase and thus an increase in the activity, which is determined via the conversion of a substrate into an optically measurable product.
Method: Ishikawa cells are sown in 96-well plates in a density of about 1×10⁴ cells/well in DMEM plus 10% of FCS. Next day, the medium is replaced by DMEM comprising 5% of oestrogen-free FCS. A further 24 hours later, the test substances are added in a concentration series in DMEM comprising 5% of oestrogen-free FCS. After incubation at 37° C. for 4 days, the activity of the alkaline phosphatase is determined. To this end, the cells are washed twice with PBS, the remaining PBS is removed, and the cells are lysed by freezing for 15 minutes at −80° C. After a thawing phase for 10 minutes at room temperature, the substrate buffer (5 mM p-nitrophenyl phosphate) is added for measurement of the alkaline phosphatase. The plates are subsequently shaken gently for a further 15 to 60 minutes, and the optical density is determined at 405 nm.
Reference: 1. LITTLEFIELD B. A., GURPIDE E., MARKIEWICZ L., MAC KINLEY B., HOCHBERG B. A simple and sensitive microtiter plate estrogen bioassay based on stimulation of alkaline phosphatase in Ishikawa cells: estrogenic action of Δ⁵ adrenal steroids. Endocrinology, 1990, 127: 2757-2762

The invention furthermore relates to the use of compounds and/or physiologically acceptable salts thereof for the preparation of a medicament (pharmaceutical composition), in particular by non-chemical methods. They can be brought into a suitable dosage form here together with at least one solid, liquid and/or semi-liquid excipient or adjuvant and optionally in combination with one or more further active compounds.

The intermediate compounds for the preparation of the compounds according to the invention can be prepared by the known general processes of the prior art, they are preferably prepared as follows:

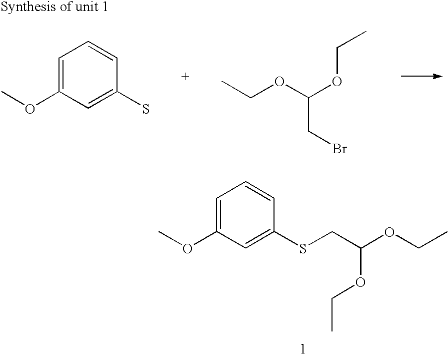

511.4 g of potassium carbonate (3.70 mol) are added in portions at room temperature to a solution of 500 g of 3-methoxythiophenol (3.57 mol) in 3 l of acetonitrile. After the mixture has been stirred for 2 h, 709.5 g of bromoacetaldehyde diethyl acetal are allowed to run in over the course of 1 h, and the mixture is stirred for a further 14 h. The solid is filtered off and rinsed with acetonitrile. The filtrate is evaporated in vacuo, dissolved in 5 l of hot petroleum ether and stirred with active carbon for 1 h. After filtration, the solution is evaporated rapidly, giving a yellow oil (933 g, 100% yield), which is confirmed as unit 1.

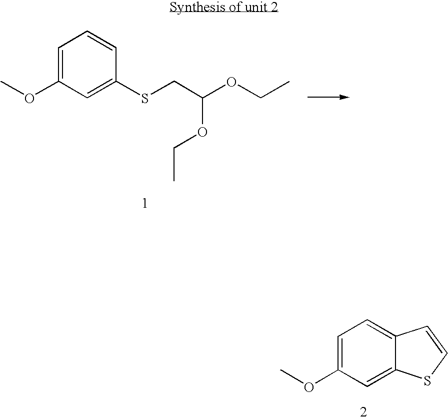

244 ml of boron trifluoride/ethyl ether complex (1.94 mol) are dissolved in 20 l of dichloromethane at 15° C. Unit 1 is subsequently dissolved in 5 l of dichloromethane at 20° C. and added dropwise over the course of 1 h. The reaction mixture is stirred for 3 h, 5 l of water are added, and the mixture is stirred for a further 1 h. The organic phase is separated off, washed with 3 l of water and 4 l of a saturated sodium hydrogencarbonate solution, dried over sodium sulfate, filtered and evaporated, giving a red oil (341 g, 75% yield), which is identified as unit 2.

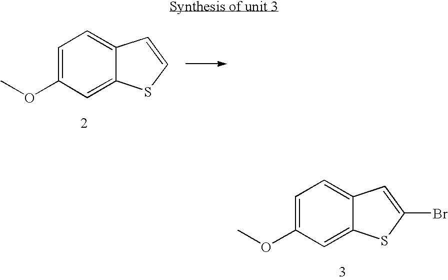

Unit 2 (73 g, 0.445 mol) is dissolved in 1 l of dichloroethane at room temperature. Toluene-4-sulfonic acid monohydrate (2 g, 10.5 mmol) is added, followed, after brief stirring, by N-bromosuccinimide (78.3 g, 0.440 mol) in small portions over the course of 30 min with gentle ice-water cooling. When the exothermic reaction is complete, the mixture is stirred at 25° C. for a further 2 h and cooled in an ice bath. The precipitated succinimide is filtered off with suction, washed with a little cold dichloroethane. The filtrate is washed with a saturated sodium hydrogencarbonate solution, dried over sodium sulfate and evaporated. The crude oil is purified over a chromatography column, giving yellow crystals (93 g, 87% yield), which can be confirmed as unit 3.

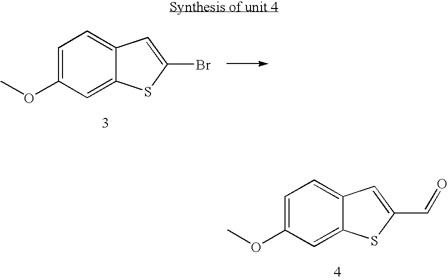

28.4 ml of an n-butyllithium solution (15% in n-hexane) are added under nitrogen at −60° C. to a solution of unit 3 (10 g, 41.13 mmol) in 250 ml of diethyl ether. After the mixture has been stirred for 30 min, 4.8 ml of N,N-dimethylformamide are added dropwise, and the mixture is slowly allowed to come to room temperature, stirred overnight, and 200 ml of water are added. The organic phase is separated off. The aqueous phase is then extracted twice with t-butyl methyl ether. The combined organic phases are dried over sodium sulfate, filtered and evaporated. The product is crystallised from hot t-butyl methyl ether and confirmed as unit 4 (5.75 g, 73% yield).

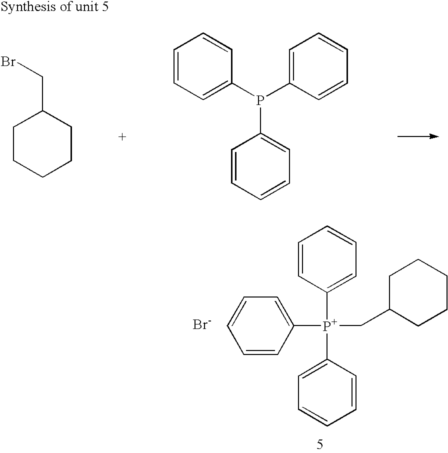

16.3 g (62.12 mmol) of triphenylphosphine are added to a solution of bromomethylcyclohexane (10 g, 56.47 mmol) in 25 ml of acetonitrile. The mixture is boiled under reflux for 48 h, cooled, 100 ml of t-butyl methyl ether are added, and the mixture is stirred for 1 h with ice-cooling. The precipitated material is filtered off and washed with portions of cold t-butyl methyl ether. The white crystals are dried at 40° C. in vacuo, giving unit 5 in the form of a white powder (18.9 g, 76% yield).

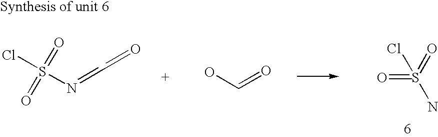

217 ml of formic acid (5.75 mol) are added at 5° C. under nitrogen and with cooling to 815 g of chlorosulfonyl isocyanate (5.76 mol). When the addition is complete, the mixture is warmed at 40° C. until completely molten, and stirring is continued overnight at room temperature. The product is taken up in toluene and concentrated in vacuo, giving unit 6 in solid form (638 g, 96% yield).

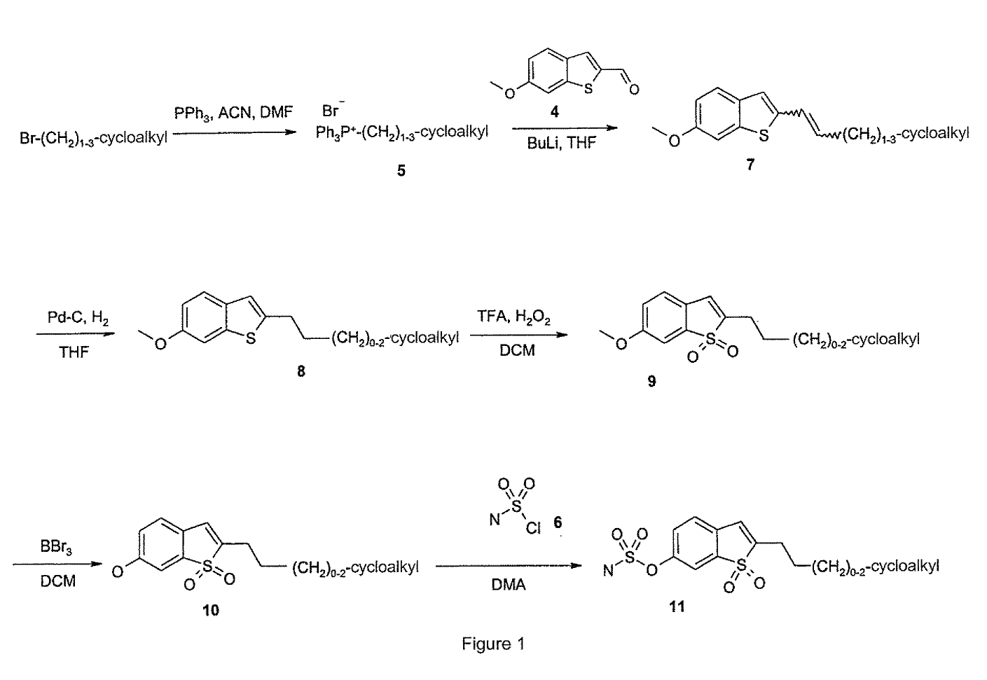

FIG. 1 shows an overview of the synthesis of compounds of the general formula (I), in which R=cycloalkyl-(CH₂)ₘ—.

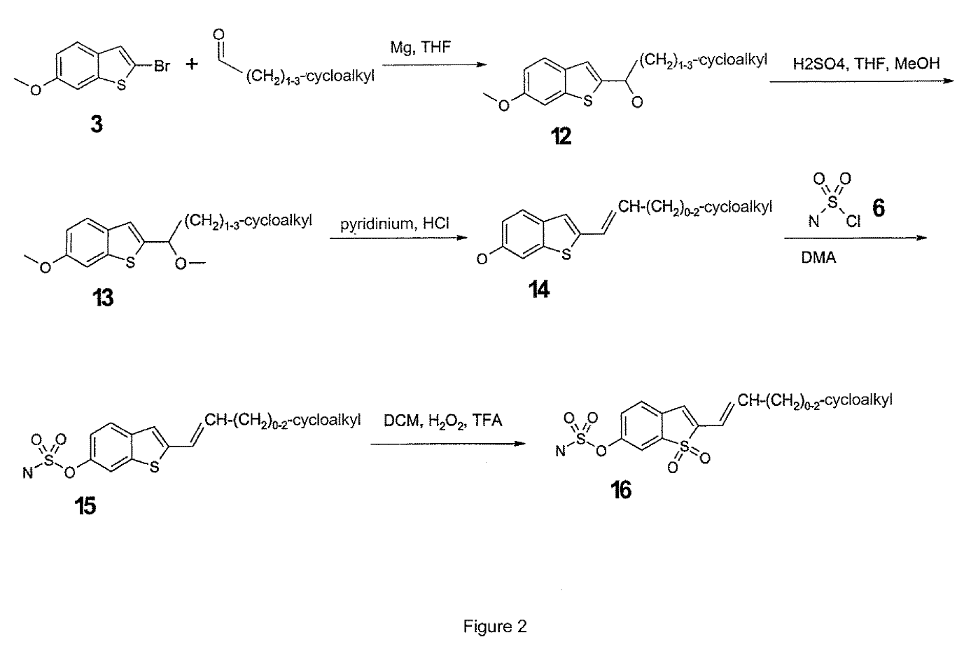

FIG. 2 shows an overview of the synthesis of compounds of the general formula (I), in which R=cycloalkylidene-(CH₂)ₙ—.

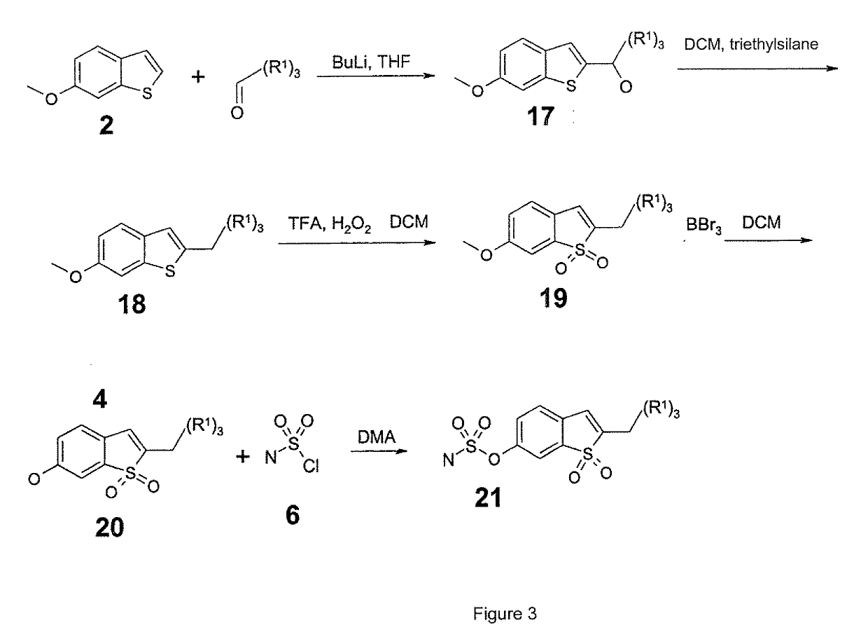

FIG. 3 shows an overview of the synthesis of compounds of the general formula (I), in which R═—(CH₂)ₙC(R¹)₃.

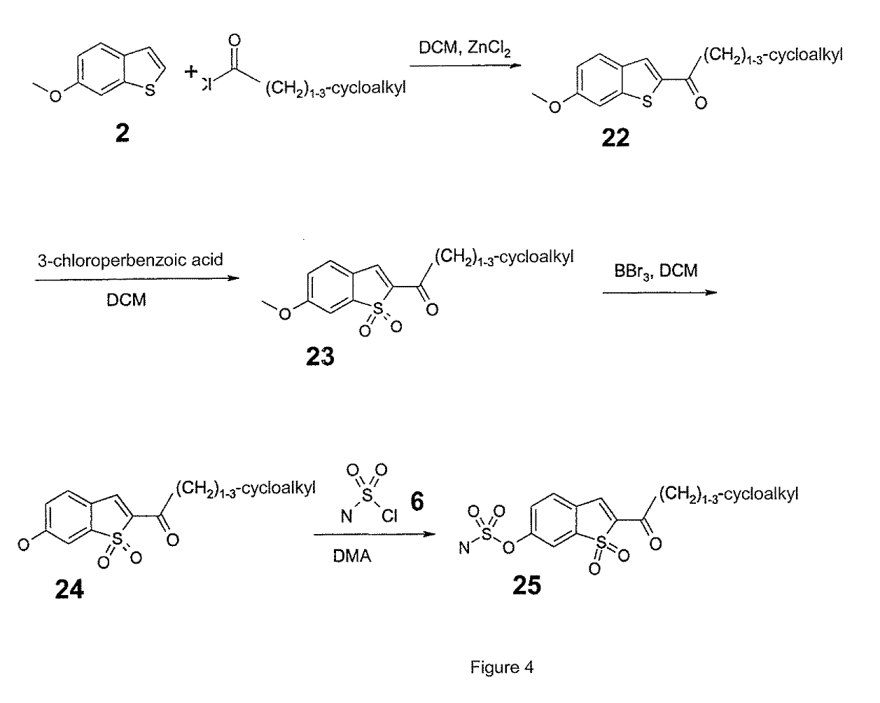

FIG. 4 shows an overview of the synthesis of compounds of the general formula (I), in which R=cycloalkyl-(CH₂)ₒCO—.

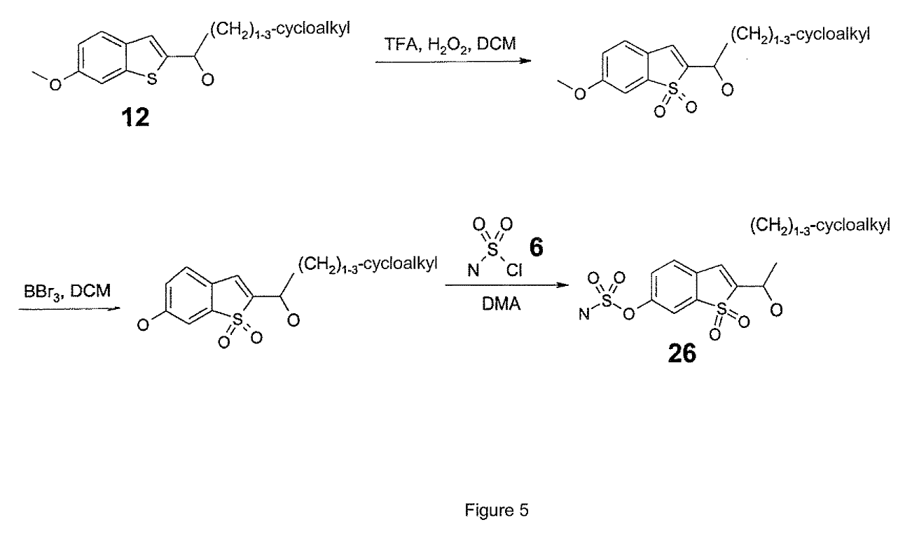

FIG. 5 shows an overview of the synthesis of compounds of the general formula (i), in which R=cycloalkyl-(CH₂)ₒC(OH)—.

The following examples show individual embodiments of the compounds according to the invention.

### EXAMPLE 1

### Preparation of 2-(2-cyclohexylethyl)-1,1-dioxo-1H-λ⁶-benzo[b]thiophen-6-yl-sulfamoyl ester

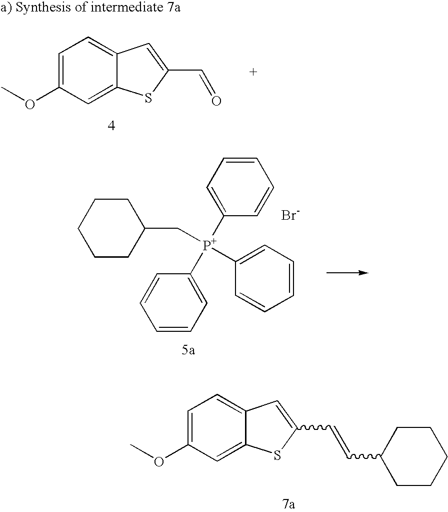

5.23 ml of butyllithium (15% solution in n-hexane) are added dropwise at −20° C. under argon to a solution of unit 5a (3.66 g, 8.32 mmol) in 15 ml of tetrahydrofuran. The mixture is stirred at 0° C. for 30 min. A solution of 800 mg of intermediate 4 (4.16 mmol) in 10 ml of THF is then added dropwise. The orange mixture is stirred overnight at room temperature, water is added, and the mixture is extracted with ethyl acetate. The organic phase is dried over sodium sulfate, filtered and evaporated in vacuo. The residue is purified over a silica-gel column, giving 900 mg of intermediate 7a (67% yield).

1H NMR (500 MHz, DMSO-d6) 1.1-1.85 (m, 11H), 3.80 (s, 3H), 5.4-7.7 (m, 5H).

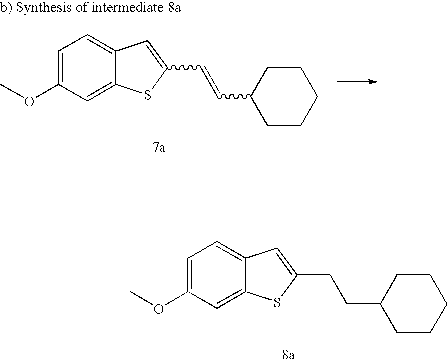

900 mg of intermediate 7a are allowed to react with 2.7 g of catalyst 5% Pd/C and 71 ml of hydrogen for 15 h at room temperature under atmospheric pressure. The catalyst is filtered off, and the filtrate is evaporated. The residue is purified over a chromatography column, giving intermediate 8a as a solid (668 mg, 84% yield).

¹H NMR (250 MHz, DMSO-d6) 0.8-1.85 (m, 15H), 3.80 (s, 3H), 6.93 (dd, 1H), 7.02 (s, 1H), 7.43 (d, 1H), 7.58 (d, 1H).

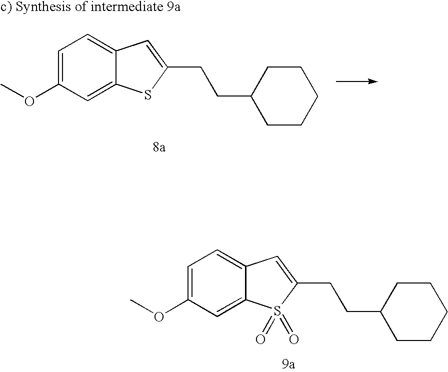

Trifluoroacetic acid (0.694 ml, 9.01 mmol) is added dropwise at 5° C. to a solution of 8a (668 mg, 2.43 mmol) in 5 ml of dichloromethane, followed by the addition of 0.795 ml (7.79 mmol) of hydrogen peroxide (30% in water) at 10° C. The mixture is stirred overnight, poured into ice-water, adjusted to pH 10-11 using sodium hydroxide solution (1 N) and extracted with dichloromethane. The organic phase is washed with a 10% iron(II) sulfate solution, dried using sodium sulfate and evaporated under reduced pressure. The moist residue is purified by chromatography, giving a white solid, which is confirmed as 9a (581 mg, 1.88 mmol, 77% yield).

¹H NMR (250 MHz, DMSO-d6) 0.8-1.85 (m, 15H), 3.85 (s, 3H), 7.12-7.22 (m, 2H), 7.4-7.5 (m, 2H).

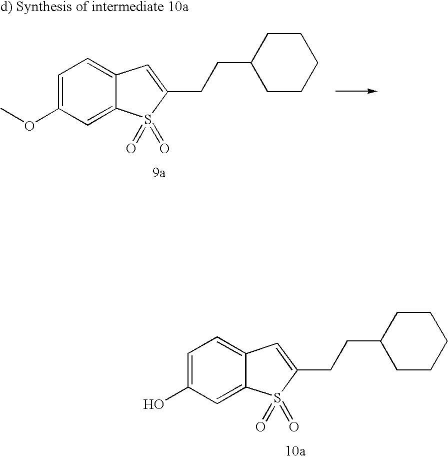

Boron tribromide (0.204 ml, 2.15 mmol) is added to a solution of 9a (550 mg, 1.795 mmol) in 5 ml of dichloromethane. The mixture is stirred overnight at room temperature, poured into ice-water. The organic phase is separated off, washed successively with water, with a saturated sodium hydrogencarbonate solution and with a saturated sodium chloride solution, dried using sodium sulfate, filtered and concentrated in vacuo. The residue is brought to crystallisation using small portions of petroleum ether and t-butyl methyl ether, filtered and dried, giving 10a as a pale solid (416 mg, 1.42 mmol, 79% yield).

¹H NMR (250 MHz, DMSO-d6) 0.8-1.85 (m, 15H), 6.98 (dd, 1H), 7.1 (m, 2H), 7.32 (d, 1H), 10.45 (s, 1H).

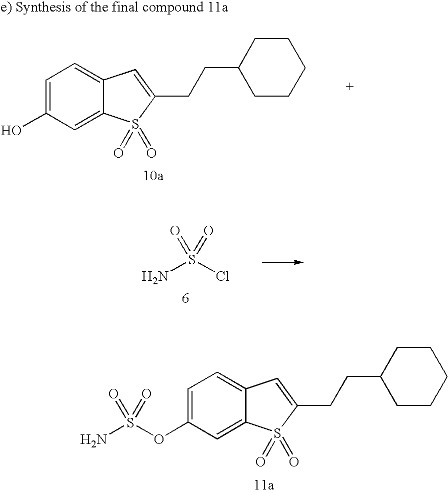

167 mg of sulfamoyl chloride 6 are added at 15° C. to a solution of 10a (383 mg, 1.31 mmol) in 2.5 ml of dimethylacetamide. The mixture is stirred at room temperature, poured into ice-water and extracted with ethyl acetate. The organic phase is separated off and washed with a saturated sodium hydrogencarbonate solution, dried using sodium sulfate, filtered and concentrated in vacuo. The residue is brought to crystallisation using small portions of petroleum ether and ethyl acetate, filtered and dried, giving 2-(2-cyclohexylethyl)-1,1-dioxo-1H-λ⁶-benzo[b]thiophen-6-ylsulfamoyl ester 11a in the form of a white solid (386 mg, 1.03 mmol, 78% yield, melting point 122° C.).

¹H NMR (250 MHz, DMSO-d6) 0.85-1.2 (m, 15H), 6.13 (s, 2H), 7.0 (t, 1H), 7.50 (m, 2H), 7.65 (t, 1H).

### EXAMPLE 2

### Preparation of 2-cycloheptylidenemethyl-1,1-dioxo-1H-λ⁶-benzo[b]thiophen-6-ylsulfamoyl ester

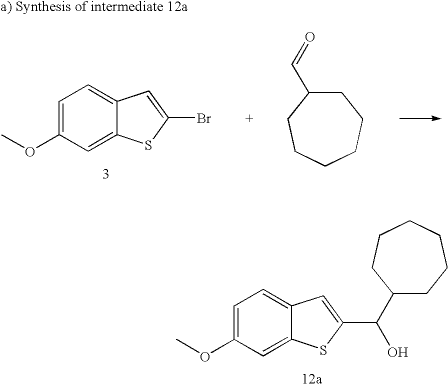

Magnesium (1.05 g, 1.1 mol) is initially introduced in 5 ml of tetrahydrofuran under argon. A solution of unit 3 (10 g, 0.041 mol) in 60 ml of tetrahydrofuran is added dropwise. The mixture is boiled under reflux for 1 h. A solution of cycloheptanecarboxaldehyde (4.4 g, 0.035 mol) in 40 ml of tetrahydrofuran is added. The mixture is boiled under reflux overnight, poured into ice-water, extracted with ethyl acetate, dried over sodium sulfate, filtered and evaporated. The crude product is chromatographed over silica gel, giving 12a (8.1 g, 93% yield) in the form of an oil.

¹H NMR (DMSO-d6) 1.1-1.8 (m, 13H), 3.8 (s, 3H), 4.6 (t, 1H), 5.6 (d, 1H), 6.9 (dd, 1H), 7.1 (s, 1H), 7.4 (d, 1H), 7.6 (d, 1H).

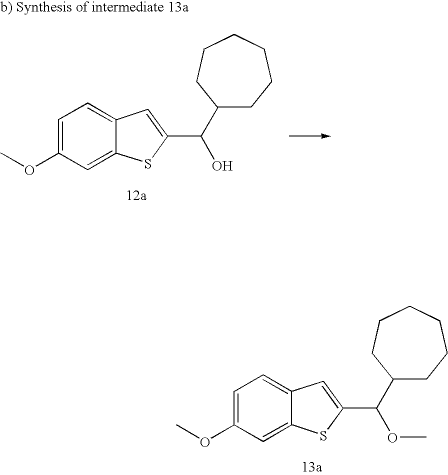

A solution of 12a (8.1 g, 27.9 mmol) in 200 ml of methanol is added dropwise to a solution of 20 ml of sulfuric acid in 100 ml of tetrahydrofuran under argon. The mixture is stirred for 30 min, poured into a saturated sodium chloride solution, extracted with ethyl acetate, washed with an ammonium chloride solution, dried over sodium sulfate, filtered and evaporated in vacuo. The residue is purified by silica-gel chromatography, giving 13a (6.7 g, 88% yield).

¹H NMR (DMSO-d6) 0.9-1.7 (m, 13H), 2.95 (s, 3H), 3.6 (s, 3H), 3.95 (d, 1H), 6.7 (2d, 1H), 6.95 (s, 1H), 7.3 (d, 1H), 7.45 (d, 1H).

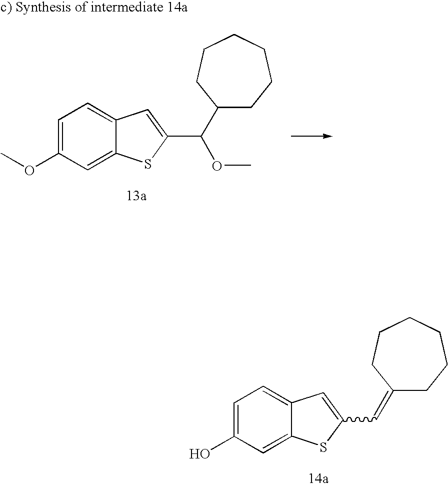

13a (5.7 g, 0.49 mol) is stirred at 160° C. for 12 h with pyridinium hydrochloride. After 2 h at room temperature, the reaction mixture is hydrolysed using a saturated ammonium chloride solution, and extracted with ethyl acetate. The organic phase is washed with a saturated sodium chloride solution, dried using sodium sulfate, filtered and concentrated in vacuo. The product is chromatographed and crystallised from dichloromethane and pentane, giving 14a (3 g, yield 53%, melting point 145° C.).

13C NMR (DMSO-d6) 26.35, 28.64, 28.74, 29.48, 31.80, 106.76, 114.62, 119.01, 122.14, 123.80, 132.09, 137.00, 140.40, 144.45, 154.83.

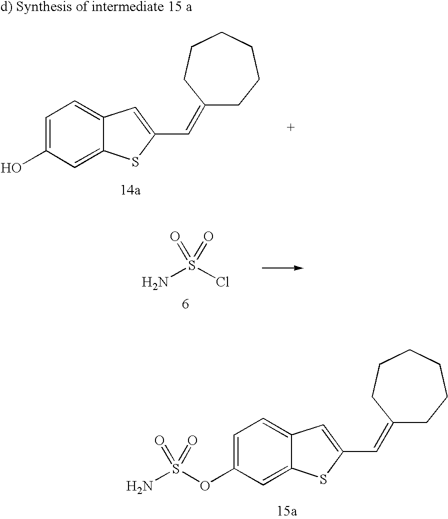

Chlorosulfonamide 6 (32 g, 0.02 mol) is added at 0° C. to a solution of 14a (2.7 g, 0.01 mol) in 15 ml of N,N-dimethylacetamide. After stirring at 0° C. for 1 h and at room temperature for 12 h, the mixture is hydrolysed using a saturated ammonium chloride solution, and extracted with ethyl acetate, dried using sodium sulfate, filtered and concentrated in vacuo. The product 15a is purified by chromatography and subsequently crystallised (1.6 g, 85% yield, melting point 130° C.).

¹H NMR (400 MHz, acetonitrile-d3) 1.57 (m, 4H), 1.68 (m, 2H), 1.77 (m, 2H), 2.45 (t, 2H), 2.7 (t, 2H), 5.96 (s, 2H), 6.52 (s, 1H), 7.2 (s, 1H), 7.28 (dd, 1H), 7.76 (m, 2H).

13C NMR (DMSO-d6) 26.27, 28.56, 28.64, 29.45, 31.94, 38.27, 115.58, 118.65, 119.74, 121.89, 123.85, 137.75, 139.15, 141.65, 146.76, 147.21.

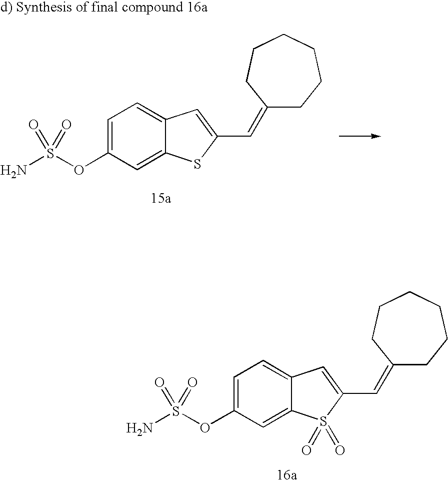

Water peroxide (0.5 ml of a 35% solution in water, 0.61 mol) is added to a solution of 15a (0.4 g, 1.2 mmol) in 30 ml of dichloromethane and 0.5 ml of trifluoroacetic acid. After stirring at room temperature for 4 h, the mixture is hydrolysed using a saturated sodium hydrogencarbonate solution, extracted with dichloromethane. The organic phase is dried using sodium sulfate, filtered and concentrated in vacuo. The crude product is purified over a chromatography column, giving 2-cycloheptylidenemethyl-1,1-dioxo-1H-λ⁶-benzo[b]thiophen-6-ylsulfamoyl ester 16a (50 mg, 10% yield) as a solid.

¹H NMR (500 MHz, acetonitrile-d3) 1.58 (m, 4H), 1.68 (m, 2H), 1.75 (m, 2H), 2.52 (t, 2H), 2.6 (t, 2H), 5.9 (s, 1H), 6.15 (s, 2H), 7.13 (s, 1H), 7.53 (m, 2H), 7.67 (s, 1H).

13C NMR (DMSO-d6) 31.49, 38.95, 44.08, 114.07, 120.75, 127.96, 131.59, 133.10, 135.90, 141.43, 147.41, 156.43, 164.00.

### EXAMPLE 3

### Preparation of 2-(2,2-dimethylpropyl)-1,1-dioxo-1H-λ⁶-benzo[b]thiophen-6-ylsulfamoyl ester

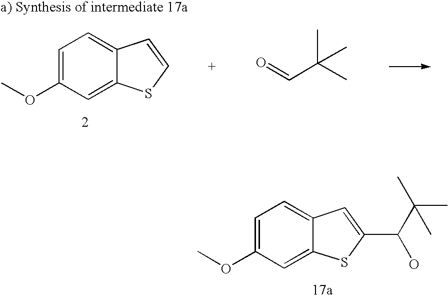

5.44 ml of a 15% butyllithium solution in tetrahydrofuran (8.67 mmol) are slowly added at −60° C. under argon to a clear solution of 1.294 g (7.88 mmol) of unit 2 in 20 ml of tetrahydrofuran. The mixture is stirred for a further 1.5 h. 747 mg of 2,2-dimethylpropionaldehyde are subsequently added at −50° C. under argon. The clear solution is allowed to come to room temperature over the course of 1 h, stirring is continued overnight, and hydrochloric acid (1 N) is slowly added to pH 1. The phases are separated, and the aqueous phase is extracted with dichloromethane. The organic phases are combined, washed with a saturated sodium chloride solution and with a saturated sodium hydrogencarbonate solution, dried using sodium sulfate, filtered and evaporated. The crystallised residue is digested with petroleum ether and filtered off with suction, giving white crystals (1.44 g, 72% yield), which can be identified as 17a.

¹H NMR (250 MHz, DMSO-d6) 0.93 (s, 9H), 3.8 (s, 3H), 4.5 (d, 1H), 5.7 (d, 1H), 6.93 (dd, 1H), 7.09 (s, 1H), 7.45 (d, 1H), 7.62 (d, 1H).

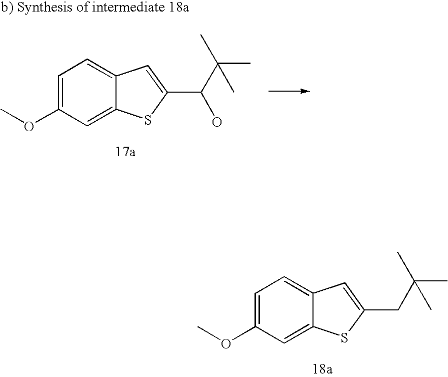

1.73 ml of triethylsilane (10.85 mmol) is slowly added dropwise with stirring and ice-bath cooling to a solution of 1.43 g of 17a (5.71 mmol) in 12 ml of dichloromethane. The mixture is stirred at 10° C. for a further 25 min and subsequently cooled to 2° C. 0.49 ml (6.28 mmol) of trifluoroacetic acid is then added dropwise. After stirring for 5 min with cooling, the mixture is allowed to come to room temperature, stirred for a further 1 h, washed twice with water, dried using sodium sulfate, filtered and evaporated. The oily colourless residue crystallised, giving 18a in the form of pale-grey crystals (1.3 g, 5.70 mmol, 99% yield).

¹H NMR (250 MHz, DMSO-d6) 0.85 (s, 9H), 2.6 (s, 2H), 3.67 (s, 3H), 6.82 (dd, 1H), 6.88 (s, 1H), 7.3 (d, 1H), 7.5 (d, 1H).

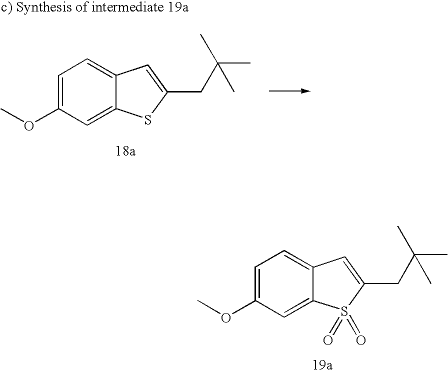

The synthesis is carried out analogously to Example 1c), giving 19a in the form of white crystals with comparable yield.

¹H NMR (400 MHz, DMSO-d6) 1.03 (s, 9H), 2.35 (s, 2H), 3.85 (s, 3H), 7.17 (dd, 1H), 7.25 (s, 1H), 7.44-7.5 (m, 2H).

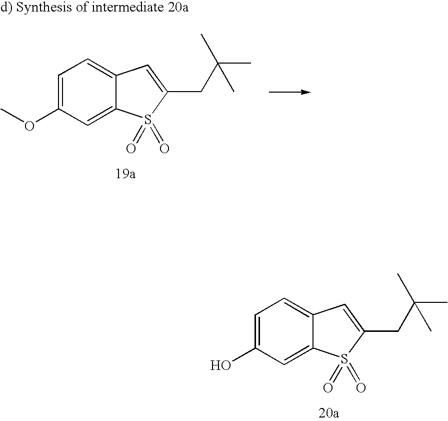

The batch is carried out analogously to the method described in Example 1 d). The intermediate 20a is isolated with comparable yield.

¹H NMR (500 MHz, DMSO-d6) 1.0 (s, 9H), 2.34 (s, 2H), 6.98 (dd, 1H), 7.1 (d, 1H), 7.2 (s, 1H), 7.35 (d, 1H), 10.45 (s, 1H).

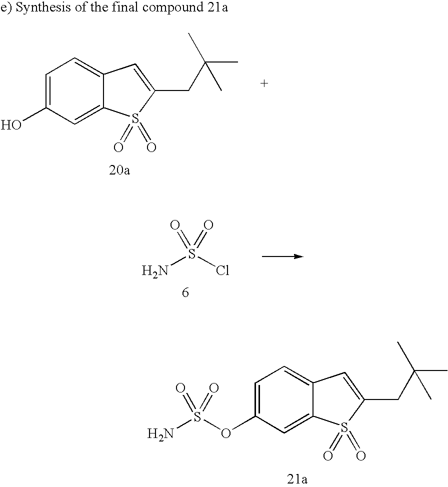

The synthesis is carried out analogously to Example 1e), giving 2-(2,2-dimethylpropyl)-1,1-dioxo-1H-λ⁶-benzo[b]thiophen-6-ylsulfamoyl ester 21 with comparable yield as a white solid.

¹H NMR (500 MHz, acetonitrile-d3) 1.05 (s, 9H), 2.45 (s, 2H), 6.16 (s, 2H), 7.12 (s, 1H), 7.52 (s, 2H), 7.65 (s, 1H);

13C NMR (400 MHz, acetonitrile-d3) 29.94, 32.38, 38.35, 116.92, 127.23, 128.93, 129.21, 131.01, 138.29, 145.02, 152.06;

Melting point 126° C.

### EXAMPLE 4

### Preparation of 2-cycloheptanecabonyl-1,1-dioxo-1H-λ⁶-benzo[b]thiophen-6-ylsulfamoyl ester

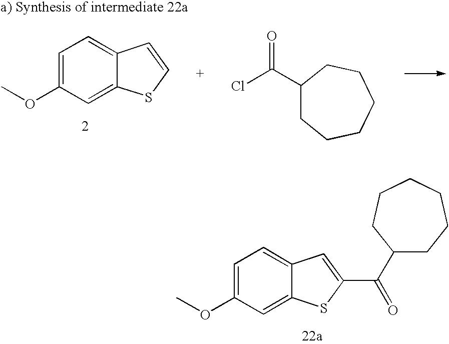

106 g (1.248 mol) of dichloromethane, 10.6 g (45.18 mmol) of unit 2 and 8.74 g (54.41 mmol) of cycloheptanecarbonyl chloride are mixed and cooled to −20° C. 20 g of tin(IV) chloride are slowly added dropwise to the solution. The mixture is stirred at −20° C. for a further 1 h, poured onto ice and 125 ml of 25% hydrochloric acid, ethyl acetate is added. The phases are separated. The aqueous phase is extracted with dichloromethane. The combined organic phases are washed with 5% sodium hydroxide solution and with water and concentrated in vacuo. The residue is purified over silica gel with a gradient from toluene and n-heptane, giving 5.85 g of product (20.3 mmol, 45% yield), which is confirmed as intermediate 22a.

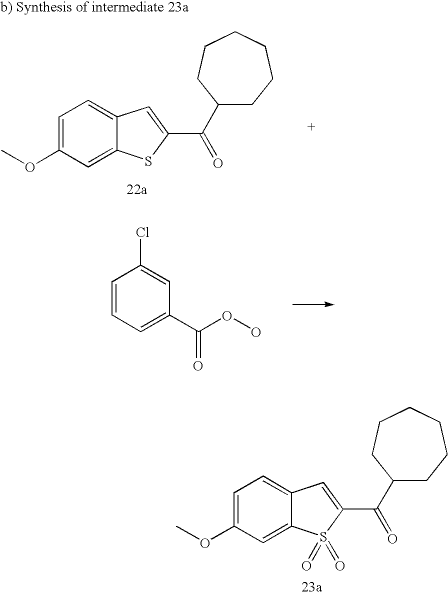

15.49 g of 3-chloroperbenzoic acid (65.98 mmol) are added to a solution, cooled to 0° C., of 8.25 g of 22a (28.61 mmol) in 78.8 g (928 mmol) of dichloromethane. The suspension is stirred further at 0° C., allowed to warm slowly to room temperature, stirred further overnight, filtered with suction and rinsed with dichloromethane. The filtrate is washed with a saturated sodium hydrogencarbonate solution, stirred with an iron(II) sulfate solution (15 g in 100 g of water) for 1 h. The phases is separated, the aqueous phase is extracted with dichloromethane. The combined organic phases are dried using sodium sulfate, filtered and concentrated, giving a yellow solid, which is confirmed as intermediate 23a (3.8 g, 41% yield).

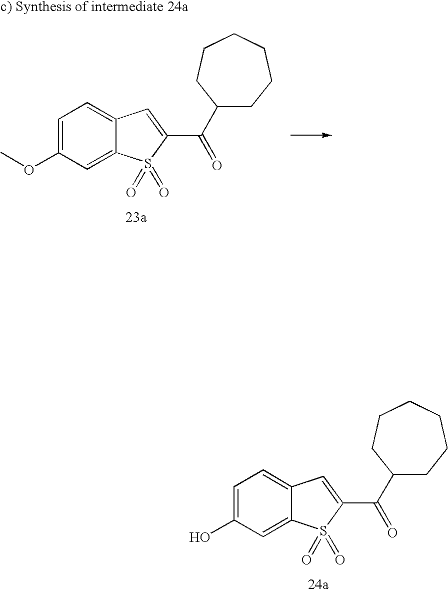

The synthesis is carried out analogously to Example 1d). A batch comprising 820 mg of intermediate 23a gives 720 mg of intermediate 24a (92% yield).

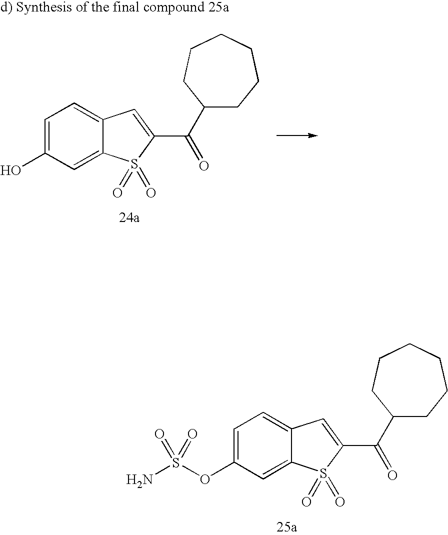

The synthesis is carried out analogously to Example 1e). 450 mg of intermediate 24a give 2-cycloheptanecarbonyl-1,1-dioxo-1H-λ⁶-benzo[b]thiophen-6-ylsulfamoyl ester 25a with comparable yield.

### EXAMPLE 5

### Preparation of 2-(cycloheptylhydroxymethyl)I-1,1-dioxo-1H-λ⁶-benzo[b]thiophen-6-ylsulfamoyl ester

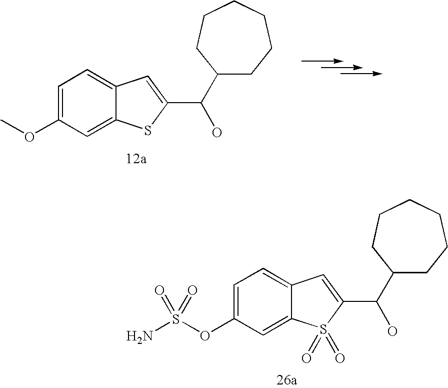

The synthesis is carried out starting from intermediate 12 in 3 steps, each analogously to Example 1c), d) and e). The two intermediates and 2-(cycloheptylhydroxymethyl)I-1,1-dioxo-1H-λ⁶-benzo[b]thiophen-6-yl-sulfamoyl ester (final compound 26a) can be isolated with comparable yields and identified.

1. Compounds of the formula (I) 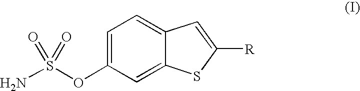
in which
R is cycloalkyl-(CH₂)ₘ—, (AA′R¹)C—(CH₂)ₙ—, cycloalkylidene-(CH₂)ₙ—, cycloalkyl-(CH₂)ₒCO— or cycloalkyl-(CH₂)ₒC(OH)—
R¹ denotes A or H,
A, A′ is each, independently of one another, alkyl having 1 to 4 C atoms
m denotes 2, 3 or 4,
n denotes 1, 2, 3 or 4,
o denotes 0, 1, 2 or 3,
stereoisomers thereof and physiologically acceptable salts and solvates thereof.

2. Compounds of the formula (I) according to claim 1, selected from the group

| Compound No. | Structure/name |
|---|---|
| ″11a″ | 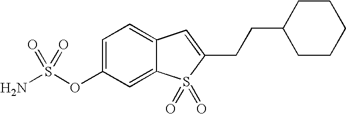 2-(2-Cyclohexylethyl)-1,1-dioxo- 1H-λ⁶-benzo[b]thiophen- 6-ylsulfamoyl ester |
| ″16a″ | 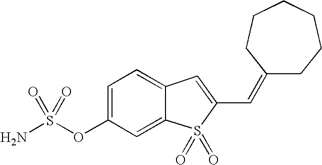 2-Cycloheptylidenemethyl-1,1-dioxo-1H-λ⁶- benzo[b]thiophen-6-ylsulfamoyl ester |
| ″21a″ | 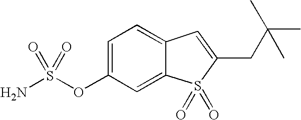 2-(2,2-Dimethylpropyl)-1,1-dioxo-1H-λ⁶-benzo[b] thiophen-6-ylsulfamoyl ester |
| ″25a″ | 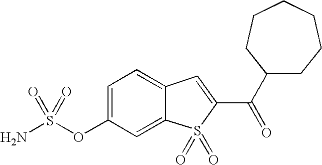 2-Cycloheptanecarbonyl-1,1-dioxo-1H-λ⁶-benzo[b] thiophen-6-ylsulfamoyl ester |
| ″26a″ | 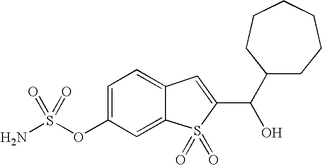 2-(Cycloheptylhydroxymethyl)1-1,1-dioxo-1H-λ⁶- benzo[b]thiophen-6-ylsulfamoyl ester |

and pharmaceutically usable derivatives, solvates, salts, tautomers and stereoisomers thereof, including mixtures thereof in all ratios.

3. A process for the preparation of the compound of the general formula (I) of claim 1, wherein
a) a compound of the general formula (II) 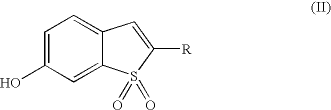
in which R has the meaning given in the general formula (I) according to claim 1,
is reacted with sulfamoyl chloride;
or
b) a compound of the general formula (III) 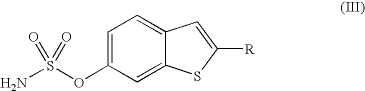
in which R has the meaning given in the general formula (I) according to claim 1,
is oxidised;
and/or
c) a compound of the general formula (I) is converted into one of its salts [lacuna] a base or acid of the formula (I).

4. Medicaments comprising at least one compound of the formula (I) according to claim 1 and/or pharmaceutically usable derivatives, salts, solvates and tautomers thereof, including mixtures thereof in all ratios, and optionally excipients and/or adjuvants.

5. A method of using compounds according to claim 1 and pharmaceutically usable derivatives, salts, solvates and tautomers thereof, including mixtures thereof in all ratios, comprising preparing a medicament for the treatment or prevention of oestrogen-dependent diseases, optionally also in combination with one or more active compounds selected from the group of the anti-oestrogens, SERMs, aromatase inhibitors, anti-androgens, lyase inhibitors, gestagens and LH-RH agonists and antagonists.

6. A method of using compounds according to claim 1 and pharmaceutically usable derivatives, salts, solvates and tautomers thereof, including mixtures thereof in all ratios, comprising preparing a medicament for the treatment or prevention of benign or malignant diseases of the breast, uterus or ovaries, optionally also in combination with one or more active compounds selected from the group of the anti-oestrogens, SERMs, aromatase inhibitors, anti-androgens, lyase inhibitors, gestagens and LH-RH agonists and antagonists.

7. A method of using compounds according to claim 1 and pharmaceutically usable derivatives, salts, solvates and tautomers thereof, including mixtures thereof in all ratios, comprising preparing a medicament for the treatment or prevention of benign or malignant diseases of the prostate or testes, in which the compounds optionally also with one or more active compounds selected from the group of the anti-oestrogens, SERMs, aromatase inhibitors, anti-androgens, lyase inhibitors, gestagens and LH-RH agonists and antagonists.

8. A method of using compounds according to claim 1 and pharmaceutically usable derivatives, salts, solvates and tautomers thereof, including mixtures thereof in all ratios, comprising preparing a medicament for the treatment or prevention of cognitive dysfunction.

9. A method of using compounds according to claim 1 and pharmaceutically usable derivatives, salts, solvates and tautomers thereof, including mixtures thereof in all ratios, comprising preparing a medicament for the treatment or prevention of immune diseases.

10. Set (kit) comprising separate packs of
(a) an effective amount of a compound according to claim 1 and/or pharmaceutically usable derivatives, solvates and tautomers thereof, including mixtures thereof in all ratios, and
(b) an effective amount of a further medicament active compound.
